(19) United States
(12) Patent Application Publication
(10) Pub. No.: US 2022/0361857 A1
(43) Pub. Date: Nov. 17, 2022

(54) SURGICAL INSTRUMENT, METHOD FOR PRODUCING A SURGICAL INSTRUMENT, AND USE OF A ROTARY JOINT FOR FORMING A CUTTING TOOL OF A SURGICAL INSTRUMENT

(71) Applicant: MORPHEUS AG, SPAICHINGEN (DE)

(72) Inventors: TIMO RACK, RIETHEIM-WEILHEIM (DE); HEIKO BLESSING, DURCHHAUSEN (DE)

(73) Assignee: MORPHEUS AG, SPAICHINGEN (DE)

(21) Appl. No.: 17/767,098

(22) PCT Filed: Oct. 8, 2020
(86) PCT No.: PCT/EP2020/078286

§ 371 Date: Apr. 7, 2022

(30) Foreign Application Priority Data
Oct. 8, 2019 (DE) 10 2019 126 965.2

(51) Int. Cl. A61B 10/02; A61B 10/06

(52) U.S. Cl. CPC A61B 10/0266; A61B 10/06; A61B 2017/320064

(57) ABSTRACT

The invention relates to a surgical instrument for separating and removing a biopsy. A suction channel for suctioning the biopsy out of a distal region in the direction of a proximal region is formed in a shaft, and/or a cutting tool is formed in one distal region or the distal region of the shaft, said cutting tool having a stationary cutting part and a counter cutting part which can be adjusted relative to the stationary cutting part about a rotational axis formed by a rotary joint wherein the cross-section of the suction channel expands in the suction direction from the distal region to the proximal region and/or the rotary joint is formed by two securing elements, each securing element being introduced from the interior to the exterior.

### CROSS-REFERENCE TO RELATED APPLICATIONS

This application is a national stage of International Application No. PCT/EP2020/078286 filed on Oct. 8, 2020, which claims priority to German Patent Application No. 10 2019 126 965.2 filed on Oct. 8, 2019, all of which are hereby incorporated by reference in their entireties.

### FIELD OF THE INVENTION

The invention relates to a surgical instrument for severing and removing a biopsy specimen (also referred to as tissue sample), wherein a suction channel for the aspiration of the biopsy specimen from a distal region in the direction of a proximal region is formed in a shaft.

Surgical instruments of this type are already known and can be designed, for example, as tissue punches. Surgical instruments of this type have a dual function. They are used firstly for severing a piece of tissue. Furthermore, however, the surgical instruments are intended not only to permit the severing but directly a removal of the tissue sample from the severing location to a collector provided for collecting the tissue sample. For this purpose, the surgical instruments have the previously mentioned suction channel which is coupled to the cutting tool in order to be able to aspirate the severed tissue sample directly from the severing location without it being necessary for the surgeon or additional staff to have to remove the tissue sample by hand.

The terms “distally” and “proximally” can each be understood with respect to a designated use situation of the surgical instrument. The term “distally” can be understood in this connection to the effect that a position remote from a hand of a user, with which the user is holding the surgical instrument, is meant thereby, and/or the term “proximally” can be understood here to the effect that a position close to a hand of a user, with which the user is holding the surgical instrument, is therefore meant thereby.

However, it has been shown that the aspiration of the biopsy specimen frequently fails in practice. A regular occurrence during the use of surgical instruments of the type mentioned at the beginning is therefore that tissue samples severed by means of the surgical instrument remain stuck during the aspiration, and the surgeon then has to interrupt the operation for the period of time of removing the blockage or of exchanging the surgical instrument. In the worst case, the removed biopsy specimen may subsequently no longer be usable for further diagnostics since it becomes unusable, for example, during removal of the blockage.

The invention also relates to a surgical instrument for severing and removing a biopsy specimen, wherein a cutting tool is formed in a distal region of the shaft, the cutting tool having a fixed cutting part and a movable counter cutting part, in particular a counter cutting part which is adjustable relative to the fixed cutting part about an axis of rotation formed by a rotary joint.

Furthermore, the invention relates to a method for producing a surgical instrument, and to the use of a rotary joint for forming a cutting tool of a surgical instrument.

### BACKGROUND OF THE INVENTION

The invention is based on the object of creating a surgical instrument of the type mentioned at the beginning with improved use properties.

This object is achieved according to the invention by a surgical instrument of the type mentioned at the beginning with the features as claimed in claim 1 and/or as claimed in claim 2 and/or as claimed in claim 7.

### SUMMARY OF THE INVENTION

In particular, according to the invention, in order to achieve the object, a surgical instrument of the type mentioned at the beginning is proposed, wherein a diameter of the cross section of the suction channel widens in the suction direction from the distal region to the proximal region. A severed biopsy specimen can therefore be transported by aspiration, for example by use of a suitable pump and/or by connection of the suction channel via a connection part to an external pump, from the distal end of the shaft through the suction channel to the proximal region, for example as far as an outlet opening. The tissue sample can finally be transferred into a tissue collector via the outlet opening. Owing to the cross-sectional widening of the suction channel, a blockage of the suction channel can be better prevented since, after the build-up of a sufficient suction force at the distal region, in particular at the cutting tool, the biopsy specimen is sucked into the suction channel, but friction arising by sliding along a wall of the suction channel can be minimized because of the cross-sectional widening of the suction channel. The biopsy here itself acts as a type of stopper which, after a threshold value of the suction force is exceeded, is released by a build-up of negative pressure and aspirated. An inside diameter of the cutting tool can therefore correspond to the diameter of the cross section of the suction channel at the distal end.

The term “cross section of the suction channel” can refer here to an extent, preferably extending perpendicularly to a longitudinal axis of the suction channel, of a cavity between two opposite walls of the suction channel.

In particular, alternatively or additionally, according to the invention, for achieving the object, a surgical instrument of the type mentioned at the beginning is proposed, wherein a rotary joint, for example the rotary joint already mentioned, is formed by two fastening elements, wherein the fastening elements are each introduced from the inside outward. In particular, in this respect on the inside refers to a suction channel inner side and/or on the outside refers to an outer side of the shaft. It can therefore be prevented that the fastening elements project too far into the suction channel and/or the biopsy specimen remains stuck to the fastening elements during the aspiration. In the case of previously known surgical instruments, the fastening elements project at least partially into the suction channel or even lead through the latter. As a result, they represent an obstacle which may lead to blockage of the suction channel by a tissue specimen. The solution according to the invention now enables the tissue sample to be aspirated without obstruction. To form subject matter likewise covered by the invention, the features of this paragraph can be combined with the features of the previously described solution to form a further solution according to the invention.

In particular, alternatively or in addition, according to the invention, in order to achieve the object, a surgical instrument of the type mentioned at the beginning is proposed, wherein the end sides of the fastening elements, preferably the end sides of the heads of the fastening elements, together with a wall of the suction channel form a planar suction channel inner side. In particular, a transition between a head and the wall can be configured to be smooth and/or continuous. It is therefore possible for blockages of the suction channel to occur due to stuck tissue samples severed by means of the cutting tool. Furthermore, it can better be prevented that the biopsy specimen is adversely affected by rubbing against a sharp-edged point of the fastening element part lying in the suction channel. To form subject matter likewise covered by the invention, the features of this paragraph can be combined with the features of the previously described solutions to form a further solution according to the invention.

The term “head of the fastening element” can refer to a fastening element part which is arranged or formed at an opposite end to the fastening element part (introduction part) introduced into an opening. The head of the fastening element can preferably at least partially have a wider diameter than the introduction part. The fastening element can be designed, for example, as a rivet, bolt, screw and/or nail.

It can be provided that a bore for one of the fastening elements or bores for the two fastening elements in a wall of the suction channel, in particular in the wall already mentioned previously, has or have a larger diameter on the inner side than on the outer side. A transition of the bore from an outer side of the wall to an inner side of the wall can take place here by means of a step. The step forms a stop for the fastening means inserted from the inside outward. Alternatively, the transition can also take place conically. A continuous transition is also possible by, for example, the transition having rounded portions. The fastening means and the bore are preferably coordinated with one another.

Advantageous refinements of the invention which, by themselves or in combination with the features of other refinements, can optionally be combined together with the features as claimed in claim 1 will be described below.

According to one advantageous refinement, it can be provided that the cross section of the suction channel widens in a widening region. In particular, the cross section of the suction channel can widen in a widening region continuously and/or rectilinearly. Therefore, after the tissue sample has been severed, first of all a sufficiently large suction force can be built up within the suction channel, which is blocked in particular initially by the tissue sample, before the tissue sample, when a threshold value is reached in which the suction force is greater than the friction force, is sucked through the suction channel. The widening region can be formed along the longitudinal direction of the suction channel at a distance from the cutting tool and/or directly adjacent to the cutting tool. A different friction force can therefore be defined in a simple manner between the tissue sample and the wall of the suction channel.

According to a further advantageous refinement, it can be provided that the suction channel has a uniformly dimensioned cross section and/or cross section which is consistent in shape upstream and/or downstream of a widening region, for example the widening region already mentioned previously. In particular, the suction channel can be formed cylindrically upstream and/or downstream of the widening region. The biopsy specimen therefore slides through the suction channel with particularly little friction. In comparison to a suction channel with consistent cross-sectional dimensions, the suction channel of the surgical instrument has the advantage that it continues still to be possible by means of it to first of all build up a suction force owing to initial blockage of the suction channel by means of the tissue sample, but, after a sufficiently high suction force is achieved and the tissue sample is transported away, the friction between the tissue sample and a wall of the suction channel can be kept as low as possible for the remaining transport distance.

According to a further advantageous refinement, it can be provided that a widening region, for example the widening region already mentioned previously, extends from a distal end of the shaft as far as at maximum to a center of the shaft, in particular as far as at maximum a third of a length of the shaft. The widening region can therefore be used to bring about an initial, intended blockage in order to build up a suction force while the biopsy specimen is located within the widening region. In the case of previously known surgical instruments with a suction channel which has an at least virtually continuously consistently wide cross section, an initial blockage is likewise brought about. However, in this case, the tissue sample may remain stuck within the suction channel during further transport, wherein then, in this state, the suction channel is sometimes closed by the biopsy sample and the biopsy sample is therefore stuck in the suction channel.

According to a further advantageous refinement, it can be provided that the end sides of the fastening elements lie in a suction channel inner side, for example the suction channel inner side already mentioned previously. In particular, the heads of the fastening elements can lie in the suction channel inner side. A simple structural solution is therefore created by means of which the abovementioned object is achieved and which is suitable for scar production of the surgical instrument.

According to a further advantageous refinement, it can be provided that the shaft has a guide rail and a sliding rail which is adjustable relative to the guide rail. Alternatively or in addition thereto, it can be provided, according to a further advantageous refinement, that the suction channel is formed by a guide rail, for example the guide rail already mentioned previously, and/or a sliding rail, for example the sliding rail already mentioned previously. In particular, the suction channel can be formed partially or completely by the guide rail and/or the sliding rail.

In order to be able better to prevent the biopsy sample that is to be removed from being rubbed or damaged as it is transported away through the suction channel, the edges of the heads of the fastening elements can be at least partially rounded and/or the end surfaces can be curved convexly.

According to a further advantageous refinement, it can be provided that the suction channel has an at least semicircular, in particular arcuate, cross section.

The invention also relates to a method for producing a surgical instrument, in particular as herein described and/or claimed, with a cutting tool which has a rotary joint. It is provided here according to the invention that a fastening element used for forming the rotary joint is introduced from the inside, i.e. a suction channel inner side, outward through a wall of a suction channel into an opening and fixed therein. In particular, an end side of the fastening element and an inner side of the wall can form a planar suction channel inner side here. The method according to the invention therefore has the advantage that production of a surgical instrument that requires few working steps and little expenditure of material is thereby possible. The outlay on costs for the production can therefore be significantly reduced compared to previously known production methods since in particular complicated post-production steps at the suction channel can be left out. For example, it is possible here to dispense with grinding a fastening element part projecting into the suction channel. The heads of the fastening elements can be inserted into a material cutout on the suction channel inner side in order preferably to form a planar surface with the wall.

For the fixing of the fastening elements introduced into the opening, the fastening elements can be welded from the outside, i.e. in particular on an outer side of the shaft and of the cutting and of the counter cutting part. The welding points and/or the fastening element portions protruding on the outer side can subsequently be ground down in order to provide a smooth outer surface so that an operator does not inadvertently cause injuries to the patient due to unground, sharp-edged regions.

The invention furthermore relates to the use of a rotary joint, which has two fastening elements inserted from the inside, for forming a cutting tool of a surgical instrument with a suction channel, in particular as herein described and claimed.

The invention will now be described in more detail with reference to a plurality of exemplary embodiments, but is not restricted to said exemplary embodiments. Further exemplary embodiments emerge from the combination of the features of individual claims or a plurality of claims with one another and/or with individual features or a plurality of features of the exemplary embodiments.

### BRIEF DESCRIPTION OF THE DRAWINGS

In the figures:

FIG. 1 shows a first possible embodiment variant of a surgical instrument for severing and removing a biopsy specimen with a cutting tool, wherein a cross section of a suction channel widens continuously in a widening region in the aspiration direction from the distal end to the proximal end,

FIG. 2 shows a top view of a horizontal longitudinal suction of the embodiment variant from FIG. 1,

FIG. 3 shows a side view of a vertical longitudinal section of the embodiment variant from FIG. 1,

FIG. 4 shows a further possible embodiment variant of a surgical instrument for severing and removing a biopsy specimen, wherein this embodiment variant likewise has a suction channel with a cross section which is at least partially increased in diameter in the aspiration direction, wherein the cutting tool has a rotary joint which has two fastening elements, wherein the fastening elements are each introduced from the inside outward such that no sharp-edged parts of the fastening elements project into the suction channel,

FIG. 5 shows a front view of the shaft of a surgical instrument according to FIG. 4, wherein the shaft is depicted without a handle part for actuating the cutting tool,

FIG. 6 shows a rear view of the shaft of a surgical instrument according to FIG. 4, wherein the shaft is depicted without a handle part for actuating the cutting tool,

FIG. 7 shows an alternatively configured distal region of a further exemplary embodiment of a surgical instrument designed according to the invention with a rotatable cutting tool,

FIG. 8 shows the surgical instrument 1 that is shown in FIG. 7 with components partially blanked out.

### DETAILED DESCRIPTION

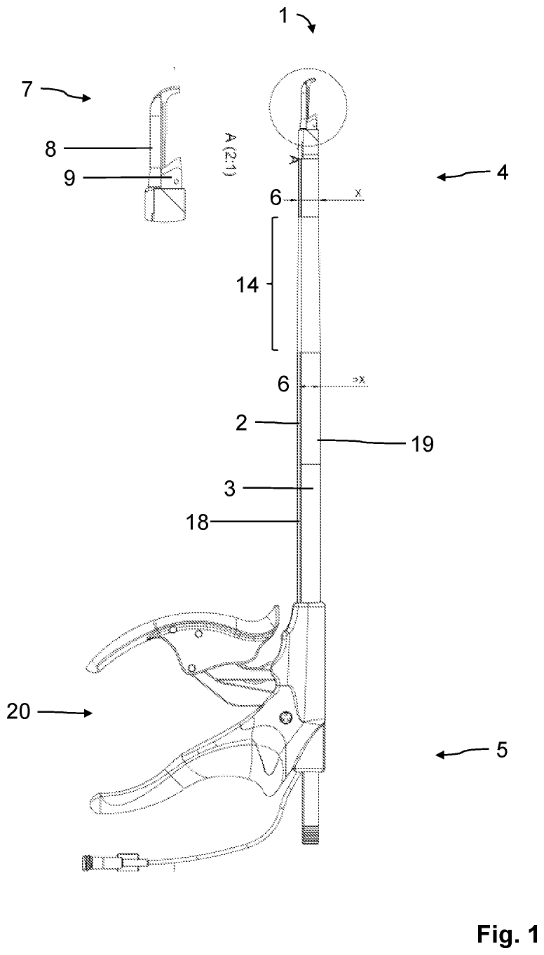

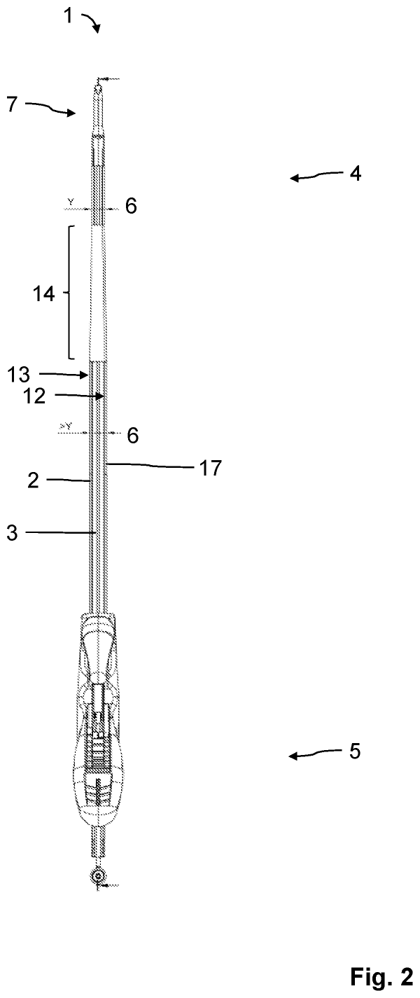

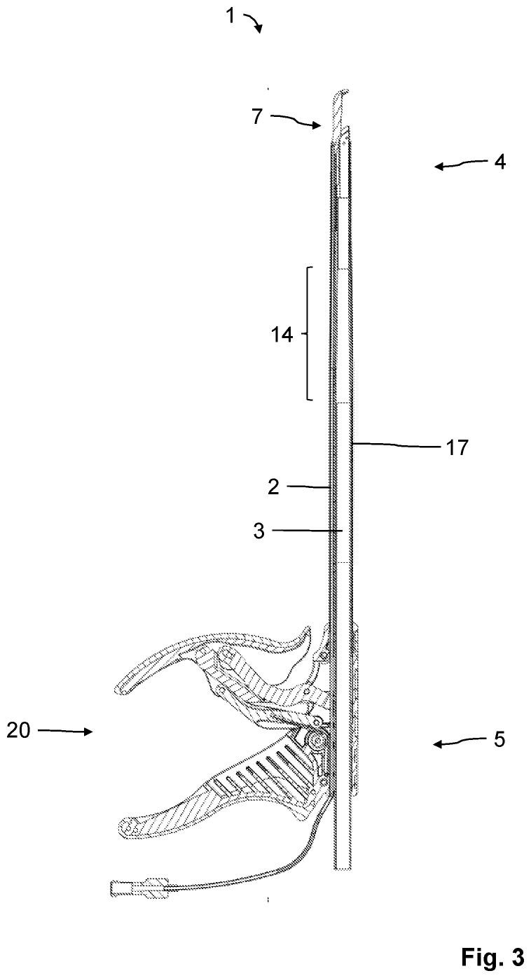

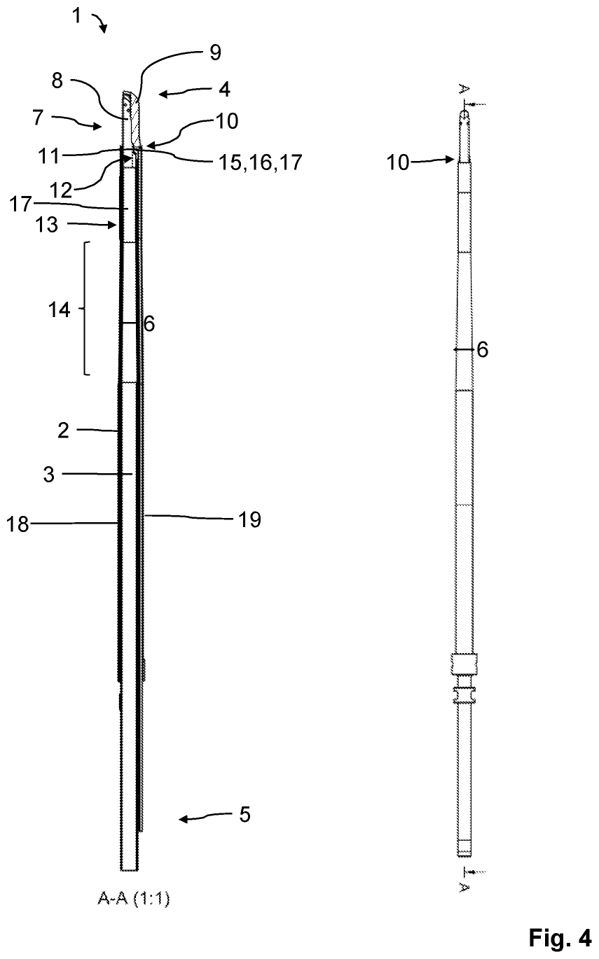

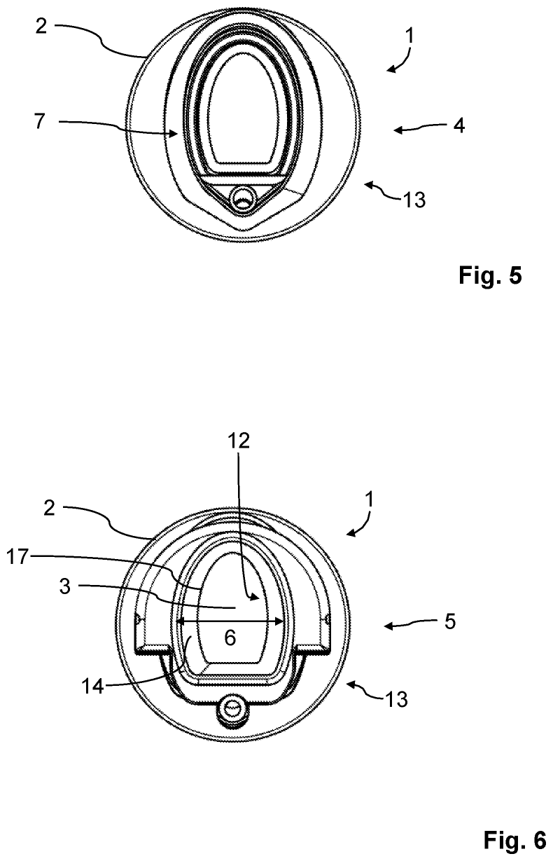

FIGS. 1-6 show possible refinements of a surgical instrument for severing and removing a biopsy specimen, i.e. in particular body material which is removed during a biopsy intervention, wherein the surgical instrument is denoted as a whole as 1.

The surgical instrument 1 can be configured, for example, as partly shown in the figures, as a tissue punch.

Such surgical instruments 1 generally have a handle part 20 by means of which a user can operate a cutting tool 8, i.e. in particular can adjust the cutting tool 8. A cutting tool 8 can basically be understood as meaning any tool of a surgical instrument 1 that is suitable and/or configured for severing tissue.

The handle part 20 is connected or connectable here fixedly to a shaft 2. The shaft is frequently constructed from multiple parts.

The shaft 2 also forms a suction channel 3 which serves to be able to transport the biopsy specimen severed by the cutting tool 8 from a distal region 4 in the direction of a proximal region 5 of the surgical instrument 1, in particular to be able to aspirate it from there. The suction operation can take place, for example, by application of a negative pressure to the suction channel 4. In particular, the surgical instrument 1 can have a connection for connecting to an external negative pressure device and a dedicated negative pressure device for generating a negative pressure.

A tissue collector can be arranged or can be arrangeable at the proximal region 5 of the shaft 2, in particular at the proximal end of the shaft 2, in order to collect the aspirated biopsy specimen.

During the aspiration of the biopsy specimen, care should be taken to ensure that the biopsy specimen is not destroyed since the intention is generally to subsequently investigate the biopsy specimen. A negative pressure applied for the aspiration of the biopsy specimen should therefore permit as gentle an aspiration as possible, for example by a pressure difference between the atmospheric pressure and the applied negative pressure not being too high.

However, if a pressure difference between the prevailing pressure and the applied negative pressure is too low, it is possible that it is not ensured that the biopsy specimen is aspirated but rather blocking of the suction channel 3 occurs.

This problem is solved in that a cross section 6 of the suction channel 3 is widened at least within a widening region 14 in the suction direction from the distal region 4 to the proximal region 5. The widening region 14 of the suction channel 3 therefore refers to the part in which there is a change in the diameter of the suction channel 3.

It is therefore possible to reduce the friction along a suction distance. At the distal end of the suction channel 3, the latter has a narrower cross section, and therefore the severed biopsy specimen lies here over a relatively large surface area against the suction channel inner side 12. After the negative pressure, in particular a vacuum, is applied, a pressure difference between prevailing external pressure and the pressure present on the biopsy specimen side facing away from the cutting tool 7 increases within the suction channel 3 until the biopsy specimen is entrained by it. The initial acceleration of the biopsy specimen is sufficient here to convey the biopsy specimen into the proximal region 5 where it is collected optionally by a tissue collector.

By means of the widening suction channel 3, the friction and/or the contact between the biopsy specimen and the wall 17 of the suction channel 3 along the suction distance is lower than at the distal region 4, i.e. in an initial position of the biopsy specimen after the latter has been severed by the cutting tool.

Depending on requirements, the surgical instrument 1 can have different cutting tools 7.

FIGS. 1-3 show an exemplary embodiment which is designed as a Kerrison punch, wherein the cutting tool 7 is designed as a bayonet. The cutting tool 7 here has a fixed cutting part 8 and a counter cutting part 9 which is adjustable axially relative thereto along a longitudinal axis.

FIGS. 4 to 6 show a second exemplary embodiment, the cutting tool 7 of which has a jaw geometry. The cutting tool 7 has a fixed cutting part 8 and a counter cutting part 9 which is adjustable relative to the fixed cutting part 8 about an axis of rotation formed by a rotary joint 10. In the case of surgical instruments 1 of this type, the previously mentioned rotary joint 10 is formed by at least one fastening element 11. If only one, for example bar-shaped, fastening element 11 is used which runs perpendicularly to the longitudinal axis of the suction channel 3 from one side of the shaft 2 to the other side, the fastening element 11 forms an obstacle within the suction channel 3. This obstacle can lead to damage to the biopsy specimen during the aspiration and/or, in the worst case, to blocking of the suction channel 3 by the biopsy specimen sticking to the fastening element 11.

Even when the rotary joint 10 is formed by two opposite fastening elements 11, the fastening elements 11 of previously known instruments project unfavorably into the suction channel 3 since, due to the simplified production, they are inserted from the outside inward. The heads 16 of the fastening elements 11 of previously known instruments therefore lie on the outer side 13 of the shaft.

In the configuration from FIGS. 4-6, it is provided that the end sides 15 of the fastening elements 11 lie in a suction channel inner side 12. Generally wider heads 16 are formed on the end side 15 in order to prevent the fastening elements 11 from slipping out of the fastening openings in the wall 17 of the suction channel 3. By means of the introduction of the fastening elements 11 from an inner side of the wall 17 outward, it is possible to prevent sharp-edged structures on the suction channel inner side 12.

The cross-sectional widening can be formed in different ways. For example, it can be continuous and/or rectilinear. It can extend over the entire suction channel 3 or only over a portion of the suction channel 3 (widening region 14).

In a refinement in which the cross-sectional widening occurs in a widening region 14, it can be provided that the suction channel 3 has a uniformly dimensioned cross section 6 and/or cross section 6 which is consistent in shape in the portion upstream and/ or downstream of the widening region 14.

As shown in FIGS. 1-6, the widening region 14 can extend from the distal end of the shaft 2 as far as at maximum to a center of the shaft 2, in particular as far as at maximum a third of a length of the shaft 2.

In the refinement from FIGS. 4-6, the end sides 15 of the fastening elements 11, i.e. preferably the end sides 15 of the heads 16 of the fastening elements 11, together with the wall 17 of the suction channel 3 form a planar suction channel inner side 12. A transition between a fastening element 11 and the wall 17 can be configured here to be smooth and/or continuous. It can therefore be prevented even better that a tissue sample remains stuck to the fastening element 11 and is thereby destroyed, and/or that a blockage occurs within the suction channel 3 in the region of the rotary joint 10. The heads of the fastening elements 11 can be at least partially recessed in a material adaptation of the wall 17.

In order to be able to even better prevent a biopsy specimen from remaining stuck to the fastening element 11, the edges of the fastening elements 11 lying on the suction channel inner side 12 can be at least partially rounded. Alternatively or additionally thereto, end surfaces 15 partially forming the suction channel inner side 12 can be curved convexly.

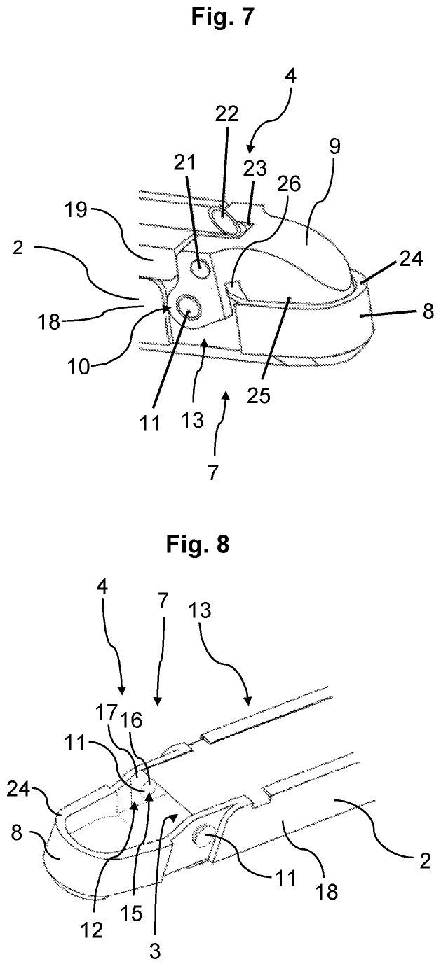

FIG. 7 and FIG. 8 show an alternatively configured distal region 4 of a further exemplary embodiment of a surgical instrument 1 designed according to the invention. In FIG. 8, some parts are blanked out in order to permit a view of the fastening elements 11 from the inside.

The cutting tool 7 has a fixed cutting part 8 and a counter cutting part 9 which is adjustable in relation thereto. The counter cutting part 9 is rotatable in relation to the cutting part 8 at the rotary joint 10. The counter cutting part 9 is connected in an articulated manner to the sliding rail 19 via the joint 21. The sliding rail 19 can slide on the guide rail 18 as previously described. If the sliding rail 19 is pushed forward, the counter cutting part 9 rotates about the rotary joint 10 into an opening of the cutting part 8. The counter cutting part 9 has merely a small amount of play within a U-shaped edge 24 of the cutting part 8 such that tissue which gets between the counter cutting part 9 and the cutting part 8 is punched out or cut off. For this purpose, the counter cutting part 9 has a cutting edge 25.

A supply line 22 through which in particular a liquid can be supplied is formed on the upper side of the sliding rail 19. If the sliding rail 19 is pushed back, a tip of the supply line 22 engages in a cutout 23 of the counter cutting part 9 such that the liquid can be introduced directly into a punching space formed between the counter cutting part 9 and the cutting part 8.

As can be seen more precisely from FIG. 8, the rotary joint 10 is formed by the fact that, on either side of the shaft 2, a respective fastening element 11 is formed which connects the fixed cutting part 8, which is positioned on the inside at this point, and the adjustable counter cutting part 9, which is positioned on the outside at this point, rotatably to one another. A recess 26 is formed in the counter cutting part 9 such that a front region of the counter cutting part 9 is no longer positioned on the outside, but rather can engage in the cutting part 8 on the inner side.

As is apparent from FIG. 8, the fastening elements 11 are introduced from the inside outward through a bore of a wall 17 of the suction channel 3. The bore here is wider on the inner side than on the outer side, and therefore the head 16 of the fastening element 11, which head is wider than the remaining region of the fastening element 11, can be completely recessed in the wall 17. An inner surface of the bore is coordinated here with a geometry of the fastening element 11.

It can be seen in FIG. 8 that the end surfaces 15 of the fastening elements 11 together with the wall 17 of the suction channel 3 form a planar suction channel inner side 12. At the same time, a transition between the head 16 and the wall 17 is configured to be smooth and continuous.

In the exemplary embodiment shown in FIG. 7 and FIG. 8, as described previously, a diameter of the cross section 6 of the suction channel 3 can be widened in the suction direction from the distal region 4 to the proximal region 5.

1-15. (canceled)

16. A surgical instrument (1) for severing and removing a biopsy specimen, comprising:
a shaft (2) having a suction channel (3) for aspiration of the biopsy specimen from a distal region (4) in the direction of a proximal region (5) of the shaft (2); and
wherein a diameter of a cross section (6) of the suction channel (3) widens in the direction from the distal region (4) to the proximal region (5).

17. A surgical instrument (1) for severing and removing a biopsy specimen, comprising:
a shaft (2) having a suction channel (3) for aspiration of the biopsy specimen from a distal region (4) in the direction of a proximal region (5) of the shaft (2); and
a cutting tool (7) formed on the distal region (4) of the shaft (2), the cutting tool having a fixed cutting part (8) and a movable counter cutting part (9); and
wherein the counter cutting part (9) is adjustable relative to the fixed cutting part (8) about an axis of rotation formed by a rotary joint (10), the rotary joint (10) being formed by two fastening elements (11); and
wherein the fastening elements (11) are each introduced from the inside outward, the inside referring to the suction channel inner side (12) of the shaft (2) and/or outward refers to toward an outer side (13) of the shaft (2).

18. The surgical instrument (1) of claim 16 wherein a cross section (6) of the suction channel (3) widens continuously and/or rectilinearly at a widening region (14).

19. The surgical instrument (1) of claim 16 wherein the suction channel (3) has a uniformly dimensioned cross section (6) upstream and/or downstream of a or the widening region (14).

20. The surgical instrument (1) of claim 16, wherein the widening region (14) extends from a distal end of the shaft (2) as far as, at maximum, a center of the shaft (2).

21. The surgical instrument (1) of claim 16, wherein end sides (15) of the fastening elements (11) lie in a suction channel inner side (12).

22. A surgical instrument (1) for severing and removing a biopsy specimen, comprising:
a shaft (2) having a suction channel (3) for aspiration of the biopsy specimen from a distal region (4) in the direction of a proximal region (5) of the shaft (2);
a cutting tool (7) formed on the distal region (4) of the shaft (2), the cutting tool (7) comprising a rotary joint (10) being formed by two fastening elements (11); and
wherein end sides (15) of heads (16) of the fastening elements (11), together with a wall (17) of the suction channel (3), form a planar suction channel inner side (12).

23. The surgical instrument (1) of claim 16, wherein the shaft (2) has a guide rail (18) and a sliding rail (19) which is adjustable relative to the guide rail (18).

24. The surgical instrument (1) of claim 16, wherein the edges of the heads (16) of the fastening elements (11) are at least partially rounded and/or the end surfaces (15) are curved convexly.

25. A method for producing a surgical instrument (1), having a cutting tool (7) which has a rotary joint (10), comprising:
introducing a fastening element (11) used for forming the rotary joint (10) from the inside outward through a wall (17) of a suction channel (3) into an opening and fixed therein such that an end side (15) of the fastening element (11) and an inner side of the wall (17) form a planar suction channel inner side (12).

26. A method for forming a cutting tool (7) of a surgical instrument (1) having a suction channel (3) comprising using a rotary joint (10), which has two fastening elements (11) inserted from the inside.

27. The surgical instrument of claim 16, wherein the cross section (6) is consistent in shape upstream and/ or downstream of a or the widening region (14).

28. The surgical instrument (1) of claim 16, wherein the widening region (14) extends from a distal end of the shaft (2) as far as, at maximum, a third of a length of the shaft (2).

29. The surgical instrument (1) of claim 16, wherein heads (16) of the fastening elements (11) lie in a suction channel inner side (12).

30. The surgical instrument (1) of claim 16, wherein the shaft (2) has a guide rail (18) and a sliding rail (19), and the suction channel (3) is formed partially or completely by the guide rail (18) and/or the sliding rail (19).

31. The surgical instrument (1) of claim 22, wherein a transition between the respective head (16) and the wall (17) is configured to be smooth and/or continuous.
